(19) United States
(12) Patent Application Publication
(10) Pub. No.: US 2024/0138160 A1
(43) Pub. Date: Apr. 25, 2024

(54) MIXED-KERNEL HETEROJUNCTION TRANSISTORS, FABRICATING METHODS, AND APPLICATIONS OF THE SAME

(71) Applicants: NORTHWESTERN UNIVERSITY, Evanston, IL (US); UNIVERSITY OF SOUTHERN CALIFORNIA, Los Angeles, CA (US)

(72) Inventors: Mark C. Hersam, Wilmette, IL (US); Xiaodong Yan, Evanston, IL (US); Justin H. Qian, Evanston, IL (US); Jiahui Ma, Los Angeles, CA (US); Vinod K. Sangwan, Evanston, IL (US); Han Wang, Los Angeles, CA (US)

(21) Appl. No.: 18/376,040

(22) Filed: Oct. 3, 2023

Related U.S. Application Data
(60) Provisional application No. 63/415,418, filed on Oct. 12, 2022.

(51) Int. Cl. H10K 19/20; A61B 5/308; A61B 5/332; A61B 5/339; A61B 5/349; H10K 19/10; H10K 85/20

(52) U.S. Cl. CPC H10K 19/20; A61B 5/308; A61B 5/332; A61B 5/339; A61B 5/349; H10K 19/10; H10K 85/221

(57) ABSTRACT

This invention in one aspect relates to a mixed-kernel heterojunction transistor, comprising a monolayer film formed of an atomically thin material, and a network of carbon nanotubes (CNTs) vertically stacked over the monolayer film to define an overlap region of the CNT network with the monolayer film, and non-overlap regions of the monolayer film and the CNT network, wherein the overlap region is a mixed-kernel van der Waals heterojunction.

### CROSS-REFERENCE TO RELATED PATENT APPLICATION

This application claims priority to and the benefit of U.S. Provisional Application No. 63/415,418, filed Oct. 12, 2022, which is incorporated herein in its entirety by reference.

### STATEMENT AS TO RIGHTS UNDER FEDERALLY-SPONSORED RESEARCH

This invention was made with government support under grant numbers DMR-1720139 and CCF-2106964 awarded by the National Science Foundation and grant numbers DE-AC02-06CH11357 and DE-NA0003525 awarded by the Department of Energy. The government has certain rights in the invention.

### FIELD OF THE INVENTION

The present invention generally relates to material science, particularly to mixed-kernel heterojunction transistors, fabricating methods, and applications of the same.

### BACKGROUND OF THE INVENTION

The background description provided herein is to present the context of the invention generally. The subject matter discussed in the background of the invention section should not be assumed to be prior art merely due to its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

The support vector machine (SVM) is a supervised machine learning algorithm that is based on quadratic programming and statistical learning theory. It is a state-of-the-art tool for classification with many applications, such as channel estimation and voice detection, due to its high efficiency. Compared to neural network classifiers, SVMs are less computationally demanding, making them more suitable for low-power applications including real-time off-grid health monitoring. For linearly separable problems, an SVM classifier identifies the hyperplane that maximizes the margin distance between two classes in the feature space. However, for practical nonlinear problems, a kernel function is typically employed to map low-dimensional inputs into a higher dimensional space, thus making linear non-separable inputs easier to classify. While a wide variety of functions can in principle be employed for this purpose, kernels are most typically derived from linear, polynomial, Gaussian, and sigmoid functions.

Although the Gaussian function, also known as the radial basis function (RBF), is the most commonly used kernel, additional kernels are often better suited for common classification tasks. For example, the Gaussian kernel has interpolation ability and is effective at identifying local properties. As a result, SVMs with Gaussian kernels have strong local learning ability (panels a-d of FIG. 5). In contrast, sigmoid kernels are better suited for identifying global characteristics but have relatively weak interpolation ability (panels e-h of FIG. 5). Therefore, mixed kernels that combine the advantages of Gaussian and sigmoid kernels often have the best classification performance for practical applications. Although SVM software implementations are highly effective for classification, they are computationally expensive and thus unsuitable for real-time applications such as continuous monitoring. Consequently, SVM hardware accelerators have been widely explored including hardware implementations of dynamically reconfigurable kernel functions using both digital and analog circuits. Analog circuits are theoretically more effective than digital architectures due to their lower power consumption and areal footprint, but even the simplest implementations of analog Gaussian functions require a significant number of circuit elements, which is an issue that is exacerbated further when implementing tunable and mixed kernel functions.

Therefore, a heretofore unaddressed need exists in the art to address the aforementioned deficiencies and inadequacies.

### SUMMARY OF THE INVENTION

In one aspect, this invention relates to a mixed-kernel heterojunction (MKH) transistor, comprising a monolayer film formed of an atomically thin material, and a network of carbon nanotubes (CNTs) vertically stacked over the monolayer film to define an overlap region of the CNT network with the monolayer film, and non-overlap regions of the monolayer film and the CNT network, wherein the overlap region is a mixed-kernel van der Waal s heterojunction.

In one embodiment, the MKH transistor further comprises a bottom gate electrode, a top gate electrode, a source electrode, a drain electrode, a first dielectric layer, a second dielectric layer, and a third dielectric layer, wherein the bottom gate electrode is formed on a substrate; the first dielectric layer is formed on the bottom gate electrode; the monolayer film is formed on the first dielectric layer; the source electrode is formed on a part of the monolayer film; the second dielectric layer is formed on the source electrode; the drain electrode is formed on the second dielectric layer on the top of the source electrode; the CNT network is formed on the drain electrode and the monolayer film to define the overlap region comprising the CNT network and the monolayer film, and the non-overlap regions each of which comprising a respective one of the CNT network and the monolayer film; the third dielectric layer is formed on the CNT network, the monolayer film and the drain electrode over the substrate; and the top gate electrode is formed on the third dielectric layer and overlapping with the overlap region and the non-overlap regions.

In one embodiment, the atomically thin material comprises a two-dimensional (2D) semiconductor material.

In one embodiment, the 2D semiconductor material comprises MoS₂, MoSe₂, WS₂, WSe₂, InSe, GaTe, black phosphorus (BP), or related 2D materials.

In one embodiment, the bottom and top gate electrodes and the source and drain electrodes comprise a same conductive material or different conductive materials.

In one embodiment, each of the bottom and top gate electrodes and the source and drain electrodes is formed of gold (Au), titanium (Ti), aluminum (Al), nickel (Ni), chromium (Cr), or other conductive materials.

In one embodiment, the first, second and third dielectric layers comprise a same dielectric material or different dielectric materials.

In one embodiment, each of the first, second and third dielectric layers is formed of Al₂O₃, HfO₂, ZrO₂, ZnO, SiO₂, or dielectrics including alumina, hafnia, or zirconia.

In one embodiment, the monolayer film comprises a monolayer MoS₂ grown by chemical vapor deposition (CVD), mechanical exfoliation, metal-organic chemical vapor deposition (MOCVD), or atomic layer deposition (ALD) as an n-type material, and the CNT network comprises solution-processed semiconducting CNT thin film as a p-type material.

In one embodiment, the overlap region in combination with the MoS₂ and CNT transistors in series in the non-overlapping regions enables highly tunable anti-ambipolar transfer characteristics.

In one embodiment, the overlap region of the MoS₂/CNT heterostructure forms a p-n junction diode with nanomaterial-enabled partial electric-field screening in the overlap region.

In one embodiment, the overlap region of the MoS₂/CNT heterostructure controls the degree of electric-field screening of the top and bottom gates.

In one embodiment, Gaussian kernel functions with tunable mean, amplitude and standard deviation are yielded under different dual-gating conditions.

In one embodiment, the Gaussian behavior is both symmetric and shows significant width tunability, which is enabled by the weak screening in the overlap region.

In one embodiment, the network density of the solution-processed CNTs is tunable over a wide range, thereby allowing precise control over the degree of screening.

In one embodiment, the network density comprises a linear density of about 7 CNTs/μm, which avoids the n-type arm in the CNT ambipolar response compared to higher CNT densities and provides the optimal level of top-gate screening.

In one embodiment, by tailoring the degree of electric-field screening through control over CNT density and overlap area, dual-gated MoS₂/CNT heterojunctions enable the MKH transistor with tunable Gaussian, sigmoid, and mixed kernel functionality.

In one embodiment, a 10 μm overlap region of the MoS₂/CNT heterojunction yields optimal sigmoid functions in comparison with smaller overlap sizes.

In one embodiment, precise control over electric-field screening in MKH transistor enables the generation of a complete set of fine-grained Gaussian, sigmoid, and mixed-kernel functions using only a single device.

In one embodiment, the MHK transistor for generating mixed kernels enables efficient and effective SVM classification for personalized arrhythmia detection from electrocardiogram (ECG) data.

In one embodiment, the MKH transistor is amenable to personalized kernels that enable arrhythmia detection accuracies approaching 95% for diverse patient profiles.

In one embodiment, in conjunction with Bayesian optimization, the MKH transistor provides effective and efficient hyperparameter searching, which further enhances classification performance.

In one embodiment, the MKH transistor is configured such that the number of circuit elements for mixed-kernel SVM is reducible by approximately two orders of magnitude, thereby enabling high classification accuracy in a scalable and energy-efficient manner.

In one embodiment, the self-aligned, semi-vertical device geometry enables to achieve a complete set of mixed Gaussian/sigmoid kernels simply by varying the biases to the top and bottom gates.

In another aspect, the invention relates to a circuitry comprising at least one MKH transistor as disclosed above.

In yet another aspect, the invention relates to a system for real-time arrhythmia detection, comprising a data acquisition for ambulatory ECG recordings; a mixed-kernel SVM circuitry for classification, wherein the mixed-kernel SVM circuitry comprises a MKH transistor device, and a mixed-kernel SVM module coupled with the data acquisition and the MKH transistor device for receiving inputs therefrom to perform arrhythmia detection; and a user interface coupled with the mixed-kernel SVM circuitry for monitoring the inputs and displaying arrhythmia types of the classification.

In one embodiment, the ambulatory ECG recordings are collected from biosensors, amplified, and preprocessed by analog-to-digital converters (ADCs).

In one embodiment, the MKH transistor device comprises a single MKH transistor to internally generate tunable Gaussian, sigmoid, and mixed kernels.

In one embodiment, the MKH transistor device comprises two MKH transistors that are separately optimized for tunable Gaussian kernels and tunable sigmoid kernels, which are then externally mixed to produce a complete set of mixed kernels.

In one embodiment, the mixing ratio is dynamically tuned by using an additive modulator based on the optimization results.

In one embodiment, hyperparameters for the mixed kernel are optimized iteratively using Bayesian optimization (BO) by maximizing the marginal likelihood of arrhythmia detection using a Gaussian process (GP), wherein the GP is a generalized Gaussian distribution that is specified by mean and covariance functions, which acts as a prior probability model.

In one embodiment, the BO process initiates from a random sample in the hyperparameter space, wherein after each BO iteration, an expected improvement (EI) serves as an acquisition function to determine the next search point in the hyperparameter space, and repeats until the search points have converged to an optimal hyperparameter combination in which the highest classification accuracy is achieved.

In one embodiment, BO-optimized mixed-kernel SVM is used for personalized arrhythmia detection.

In one embodiment, in an SVM hardware implementation, the scalability of the MKH transistors comprises an n×n kernel matrix.

In one embodiment, each kernel cell in the n×n kernel matrix needs to generate a complete set of mixed kernels by only two MKH transistors.

In one embodiment, the MKH approach has reduced power consumption compared to CMOS for mixed-kernel SVM hardware. The tunable Gaussian, sigmoid, and mixed kernels generated by MKH transistors only require tens of nanowatts of power.

These and other aspects of the present invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows mixed-kernel heterojunction transistor schematic, structure, and performance according to embodiments of the invention. Panel a: Partial schematic of the device geometry showing the MoS₂/CNT overlap region that controls the degree of electric-field screening of the top and bottom gates. Panel b: Full schematic of the completed device. Panel c: Optical microscopy image of the fabricated device with the MoS₂, CNT, and MoS₂/CNT overlap regions labeled. Panel d: I_{D}-V_{D} curve of the device with both the top and bottom gates grounded. Panel e: I_{D}-V_{BG} curves of the device in Gaussian operation mode showing tunable mean. Panel f: I_{D}-V_{BG} curves of the device in Gaussian operation mode showing tunable amplitude. Panel g: I_{D}-V_{BG} curves of the device in Gaussian operation mode showing tunable standard deviation. Panel h: I_{D}-V_{TG} curves of the device in tunable sigmoid operation mode. The bottom gate is held at a constant bias V_{BG}=5 V. Panel i: I_{D}-V_{TG} curves of the device showing tunable mixed-kernel generation.

FIG. 2 shows arrhythmia detection using single-device-generated mixed kernels according to embodiments of the invention. Panel a: Schematic of the mixed-kernel SVM circuitry and user interface design for arrhythmia detection. Ambulatory ECG recordings are collected from biosensors, and an optimal mixed kernel is used to identify six different types of arrhythmia. Panel b: Typical heartbeat ECG dataset. Panel c: Six arrhythmia types: normal beat (N), atrial premature beat (A), premature ventricular contraction (V), paced beat (/), left bundle branch block beat (L), and right bundle branch block beat (R). Panel d: Photograph of the circuit board used to implement the mixed-kernel SVM. Panel e: Classification accuracy for different mixed kernels: (1) 100% sigmoid+0% Gaussian; (2) 80% sigmoid+20% Gaussian; (3) 50% sigmoid+50% Gaussian; (4) 20% sigmoid+80% Gaussian; (5) 0% sigmoid+100% Gaussian.

FIG. 3 shows Bayesian optimization of personalized mixed kernels according to embodiments of the invention. Panels a-c: Bayesian optimization of the Gaussian kernel hyperparameter, sigmoid kernel hyperparameter, and mixing ratio after 5, 15, and 25 iterations, respectively. Panel d: Schematic of personalized SVM classification enabled by using two MKH transistors to generate a complete set of mixed kernels. Panel e: Arrhythmia detection accuracy comparison for SVMs using only a Gaussian kernel, only a sigmoid kernel, or a personalized mixed kernel for 10 different patient data sets. Panel f: Overall statistics derived from 100 different patient data sets showing that the personalized mixed kernel has higher arrhythmia detection accuracy than SVMs using only a Gaussian kernel or only a sigmoid kernel.

FIG. 4 shows mixed kernel circuit complexity comparison with conventional CMOS according to embodiments of the invention. Panel a: Comparison of the circuit blocks needed to generate a complete set of mixed kernels for CMOS and MKH implementations. CMOS circuits require many building blocks compared to only two MKH transistors. Panel b: Comparison of the number of devices needed to generate a complete set of mixed kernels for CMOS and MKH implementations. CMOS circuits require >100 devices compared to only two MKH transistors. Panel c: Schematic showing an n×n kernel matrix that is required to perform kernel computation for two n-input vectors in a hardware-level SVM implementation. Panel d: Comparison of the number of devices needed to implement an n×n kernel matrix for CMOS and MKH implementations.

FIG. 5 shows characterization of mixed-kernel heterojunction transistors according to embodiments of the invention. Panel a: two classes of data are randomly generated with a concentric distribution shape in feature space. Panel b: three Gaussian kernel functions generated by the mixed-kernel heterojunction (MKH) transistor. Panel c: a Gaussian kernel is suitable to classify the data from panel a by utilizing the geometric discrepancies around the center and tail areas in a Gaussian curve. Following application of an optimized Gaussian kernel, the data can be classified linearly with a hyperplane in the resulting three-dimensional Hilbert feature space. Panel d: A receiver operating characteristic (ROC) plot illustrates the diagnostic ability of the binary support vector machine (SVM) classifier upon application of the different Gaussian kernels in panel b. A curve higher above the random decision performance line with a larger area-under-the-curve (AUC) value represents better classification performance. Panel e: Two classes of data are randomly distributed in a diagonal way, where one class mainly occupies the first and third quadrants and the other class mainly occupies the second and fourth quadrants. Panel f: Three sigmoid kernel functions generated by the MKH transistor. (panel g) A sigmoid kernel is suitable for classifying the data from panel e. Panel h: ROC plot of SVM classification with the different sigmoid kernels from panel f.

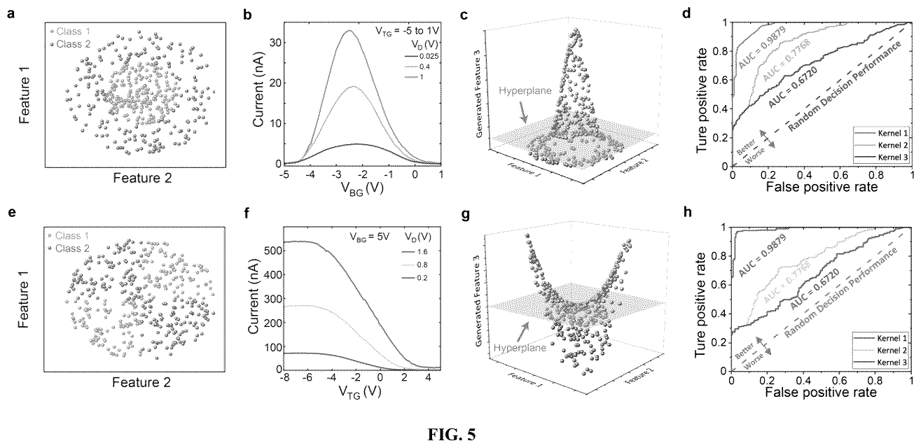

FIG. 6 shows drain current versus V_{TG} for different carbon nanotube (CNT) densities in MKH transistors according to embodiments of the invention.

FIG. 7 shows drain current versus V_{TG} for different CNT overlap region lengths in MKH transistors according to embodiments of the invention.

FIG. 8 shows (panel a) Gaussian fitting of representative curves from panel g of FIG. 1, (panel b) sigmoid fitting of representative curves from panel h of FIG. 1, and (panel c) mixed-kernel fitting of a representative curve from panel i of FIG. 1, according to embodiments of the invention.

### DETAILED DESCRIPTION OF THE INVENTION

The invention will now be described more fully hereinafter with reference to the accompanying drawings, in which exemplary embodiments of the invention are shown. However, this invention may be embodied in many different forms and should not be construed as limited to the embodiments set forth herein. Rather, these embodiments are provided so that this specification will be thorough and complete and fully convey the invention's scope to those skilled in the art. Like reference numerals refer to like elements throughout.

The terms used in this specification generally have their ordinary meanings in the art, within the context of the invention, and in the specific context where each term is used. Certain terms used to describe the invention are discussed below, or elsewhere in the specification, to provide additional guidance to the practitioner regarding the description. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, or section without departing from the invention's teachings.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. Therefore, the exemplary terms “below” or “beneath” can encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

Advances in algorithms and low-power computing hardware have led to increasing interest in machine learning for off-grid medical data classification and diagnosis. Electrocardiogram (ECG) interpretation is one such application that currently requires significant time from trained medical staff. Although support vector machine (SVM) algorithms for ECG classification show high classification accuracy, hardware implementations for edge applications are currently impractical due to the complexity and high power consumption in SVM kernels from conventional complementary metal-oxide semiconductors (CMOS) circuits. Here we report reconfigurable mixed-kernel transistors based on dual-gated van der Waals heterojunctions that can generate Gaussian, sigmoid, and mixed Gaussian/sigmoid functions for analog SVM kernel applications. The resulting heterojunction-generated kernels are employed for arrhythmia detection from ECG signals with high classification accuracy. In addition to achieving superior accuracy compared to standard radial basis function kernels, the reconfigurable nature of mixed-kernel heterojunction transistors also allows for personalized detection using Bayesian optimization. Since a single mixed-kernel heterojunction device can generate the equivalent transfer function of a CMOS circuit comprised of dozens of transistors, this approach can achieve ultralow power hardware kernel generators with broad applicability to SVM classification applications.

In one aspect, this invention relates to a mixed-kernel heterojunction (MKH) transistor, comprising a monolayer film formed of an atomically thin material, and a network of carbon nanotubes (CNTs) vertically stacked over the monolayer film to define an overlap region of the CNT network with the monolayer film, and non-overlap regions of the monolayer film and the CNT network, wherein the overlap region is a mixed-kernel van der Waals heterojunction.

In one embodiment, the MKH transistor further comprises a bottom gate electrode, a top gate electrode, a source electrode, a drain electrode, a first dielectric layer, a second dielectric layer, and a third dielectric layer, wherein the bottom gate electrode is formed on a substrate; the first dielectric layer is formed on the bottom gate electrode; the monolayer film is formed on the first dielectric layer; the source electrode is formed on a part of the monolayer film; the second dielectric layer is formed on the source electrode; the drain electrode is formed on the second dielectric layer on the top of the source electrode; the CNT network is formed on the drain electrode and the monolayer film to define the overlap region comprising the CNT network and the monolayer film, and the non-overlap regions each of which comprising a respective one of the CNT network and the monolayer film; the third dielectric layer is formed on the CNT network, the monolayer film and the drain electrode over the substrate; and the top gate electrode is formed on the third dielectric layer and overlapping with the overlap region and the non-overlap regions.

In one embodiment, the atomically thin material comprises a two-dimensional (2D) semiconductor material.

In one embodiment, the 2D semiconductor material comprises MoS₂, MoSe₂, WS₂, WSe₂, InSe, GaTe, black phosphorus (BP), or related 2D materials.

In one embodiment, the bottom and top gate electrodes and the source and drain electrodes comprise a same conductive material or different conductive materials.

In one embodiment, each of the bottom and top gate electrodes and the source and drain electrodes is formed of gold (Au), titanium (Ti), aluminum (Al), nickel (Ni), chromium (Cr), or other conductive materials.

In one embodiment, the first, second and third dielectric layers comprise a same dielectric material or different dielectric materials.

In one embodiment, each of the first, second and third dielectric layers is formed of Al₂O₃, HfO₂, ZrO₂, ZnO, SiO₂, or dielectrics including alumina, hafnia, or zirconia.

In one embodiment, the monolayer film comprises a monolayer MoS₂ grown by chemical vapor deposition (CVD), mechanical exfoliation, metal-organic chemical vapor deposition (MOCVD), or atomic layer deposition (ALD) as an n-type material, and the CNT network comprises solution-processed semiconducting CNT thin film as a p-type material.

In one embodiment, the overlap region in combination with the MoS₂ and CNT transistors in series in the non-overlapping regions enables highly tunable anti-ambipolar transfer characteristics.

In one embodiment, the overlap region of the MoS₂/CNT heterostructure forms a p-n junction diode with nanomaterial-enabled partial electric-field screening in the overlap region.

In one embodiment, the overlap region of the MoS₂/CNT heterostructure controls the degree of electric-field screening of the top and bottom gates.

In one embodiment, Gaussian kernel functions with tunable mean, amplitude and standard deviation are yielded under different dual-gating conditions.

In one embodiment, the Gaussian behavior is both symmetric and shows significant width tunability, which is enabled by the weak screening in the overlap region.

In one embodiment, the network density of the solution-processed CNTs is tunable over a wide range, thereby allowing precise control over the degree of screening.

In one embodiment, the network density comprises a linear density of about 7 CNTs/μm, which avoids the n-type arm in the CNT ambipolar response compared to higher CNT densities and provides the optimal level of top-gate screening.

In one embodiment, by tailoring the degree of electric-field screening through control over CNT density and overlap area, dual-gated MoS₂/CNT heterojunctions enable the MKH transistor with tunable Gaussian, sigmoid, and mixed kernel functionality.

In one embodiment, a 10 μm overlap region of the MoS₂/CNT heterojunction yields optimal sigmoid functions in comparison with smaller overlap sizes.

In one embodiment, precise control over electric-field screening in MKH transistor enables the generation of a complete set of fine-grained Gaussian, sigmoid, and mixed-kernel functions using only a single device.

In one embodiment, the MHK transistor for generating mixed kernels enables efficient and effective SVM classification for personalized arrhythmia detection from electrocardiogram (ECG) data.

In one embodiment, the MKH transistor is amenable to personalized kernels that enable arrhythmia detection accuracies approaching 95% for diverse patient profiles.

In one embodiment, in conjunction with Bayesian optimization, the MKH transistor provides effective and efficient hyperparameter searching, which further enhances classification performance.

In one embodiment, the MKH transistor is configured such that the number of circuit elements for mixed-kernel SVM is reducible by approximately two orders of magnitude, thereby enabling high classification accuracy in a scalable and energy-efficient manner.

In one embodiment, the self-aligned, semi-vertical device geometry enables to achieve a complete set of mixed Gaussian/sigmoid kernels simply by varying the biases to the top and bottom gates.

In another aspect, the invention relates to a circuitry comprising at least one MKH transistor as disclosed above.

In yet another aspect, the invention relates to a system for real-time arrhythmia detection, comprising a data acquisition for ambulatory ECG recordings; a mixed-kernel support vector machine (SVM) circuitry for classification, wherein the mixed-kernel SVM circuitry comprises a mixed-kernel heterojunction (MKH) transistor device, and a mixed-kernel SVM module coupled with the data acquisition and the MKH transistor device for receiving inputs therefrom to perform arrhythmia detection; and a user interface coupled with the mixed-kernel SVM circuitry for monitoring the inputs and displaying arrhythmia types of the classification.

In one embodiment, the ambulatory ECG recordings are collected from biosensors, amplified, and preprocessed by analog-to-digital converters (ADCs).

In one embodiment, the MKH transistor device comprises a single MKH transistor to internally generate tunable Gaussian, sigmoid, and mixed kernels.

In one embodiment, the MKH transistor device comprises two MKH transistors that are separately optimized for tunable Gaussian kernels and tunable sigmoid kernels, which are then externally mixed to produce a complete set of mixed kernels.

In one embodiment, the mixing ratio is dynamically tuned by using an additive modulator based on the optimization results.

In one embodiment, hyperparameters for the mixed kernel are optimized iteratively using Bayesian optimization (BO) by maximizing the marginal likelihood of arrhythmia detection using a Gaussian process (GP), wherein the GP is a generalized Gaussian distribution that is specified by mean and covariance functions, which acts as a prior probability model.

In one embodiment, the BO process initiates from a random sample in the hyperparameter space, wherein after each BO iteration, an expected improvement (EI) serves as an acquisition function to determine the next search point in the hyperparameter space, and repeats until the search points have converged to an optimal hyperparameter combination in which the highest classification accuracy is achieved.

In one embodiment, BO-optimized mixed-kernel SVM is used for personalized arrhythmia detection.

In one embodiment, in an SVM hardware implementation, the scalability of the MKH transistors comprises an n×n kernel matrix.

In one embodiment, each kernel cell in the n×n kernel matrix needs to generate a complete set of mixed kernels by only two MKH transistors.

In one embodiment, the MKH approach has reduced power consumption compared to CMOS for mixed-kernel SVM hardware. The tunable Gaussian, sigmoid, and mixed kernels generated by MKH transistors only require tens of nanowatts of power.

The invention may find applications in support vector machines, probabilistic neural network circuits, Bayesian inference circuits, edge computing circuits, off-grid classifiers, high throughput local data analysis, and analog electronics

Among other things, the invention provides at least the following advantages.

The support vector machine (SVM) is a supervised machine learning algorithm that is based on quadratic programming and statistical learning theory. Compared to neural network classifiers, SVMs are less computationally demanding, making them more suitable for low-power applications including real-time off-grid health monitoring. Although the Gaussian function, also known as the radial basis function (RBF), is the most commonly used kernel, mixed kernels that combine the advantages of Gaussian and sigmoid kernels often have the best classification performance for practical applications. SVM software implementations are highly effective for classification, but they are computationally expensive and thus unsuitable for real-time applications such as continuous monitoring. Analog circuits are theoretically more effective than digital architectures due to their lower power consumption and reduced areal footprint. However, even the simplest implementations of analog Gaussian functions require a significant number of CMOS-based circuit elements. This issue is further exacerbated further when implementing tunable and mixed kernel functions.

Dual-gated mixed-kernel heterojunction (MKH) transistors are built using monolayer MoS₂ grown by chemical vapor deposition (CVD) as the n-type material and solution-processed semiconducting carbon nanotube (CNT) thin film as the p-type material. Precise control over electric-field screening in MKH transistors enables the generation of a complete set of fine-grained Gaussian, sigmoid, and mixed-kernel functions using only a single device. Other demonstrations of transistor-based kernels lack hyperparameter tunability and the ability to generate mixed kernels. In conjunction with Bayesian optimization, MKH transistors provide effective and efficient hyperparameter searching, which further enhances classification performance. Compared with equivalent kernel generation using conventional CMOS circuits, our MKH transistors reduce the number of circuit elements for mixed-kernel SVM by approximately two orders of magnitude, thereby enabling high classification accuracy in a scalable and energy-efficient manner.

These and other aspects of the invention are further described below. Without intent to limit the scope of the invention, exemplary instruments, apparatus, methods, and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### EXAMPLE

### Reconfigurable Mixed-Kernel Heterojunction Transistors for Personalized Support Vector Machine Classification

Advances in algorithms and low power computing hardware have led to increasing interest in machine learning for off-grid medical data classification and diagnosis. Electrocardiogram (ECG) interpretation is one such application that currently requires significant time from trained medical staff Although support vector machine (SVM) algorithms for ECG classification show high classification accuracy, hardware implementations for edge applications are currently impractical due to the complexity and high power consumption needed for SVM kernel optimization using conventional complementary metal-oxide semiconductors (CMOS) circuits. Here we report reconfigurable mixed-kernel transistors based on dual-gated van der Waals heterojunctions that can generate Gaussian, sigmoid, and mixed Gaussian/sigmoid functions for analog SVM kernel applications. The resulting heterojunction-generated kernels are employed for arrhythmia detection from ECG signals with high classification accuracy. In addition to achieving superior accuracy compared to standard radial basis function kernels, the reconfigurable nature of mixed-kernel heterojunction transistors also allows for personalized detection using Bayesian optimization. Since a single mixed-kernel heterojunction device can generate the equivalent transfer function of a CMOS circuit comprised of dozens of transistors, this approach can achieve ultralow power hardware kernel generators with broad applicability to SVM classification applications.

Specifically, in this exemplary study dual-gated mixed-kernel heterojunction (MKH) transistors are disclosed by using monolayer chemical vapor deposition (CVD) MoS₂ as the n-type material and solution-processed semiconducting carbon nanotubes (CNT) as the p-type material. Precise control over electric-field screening in MKH transistors enables the generation of a complete set of fine-grained Gaussian, sigmoid, and mixed-kernel functions using only a single device. In conjunction with Bayesian optimization, MKH transistors provide effective and efficient hyperparameter searching, which further enhances classification performance. By taking into account user diversity through personalized hyperparameter optimization, precise arrhythmia detection can be derived from electrocardiogram (ECG) data. Lastly, we demonstrate the scalability of our MKH transistors in an SVM hardware implementation composed of an n×n kernel matrix. Compared with equivalent kernel generation using conventional CMOS circuits, our MKH transistors reduce the number of circuit elements for mixed-kernel SVM by approximately two orders of magnitude, thereby enabling high classification accuracy in a scalable and energy-efficient manner.

### Materials and Methods

Mixed-kernel heterojunction transistor fabrication: Photolithography was performed on a Heidelberg MLA150 Maskless Aligner with an exposure wavelength of 375 nm and an exposure dosage of 750 mJ cm⁻², and on a Suss MABA6 Mask Aligner with an exposure wavelength of 365 nm and an exposure intensity of 10 mW cm⁻². Negative resist (NR9-1000PY, Futurrex) was used on undoped Si substrates, and baked for 1 min at 150° C. pre-exposure, and 1 min at 100° C. post-exposure. Resist development was performed in 1:1 diluted RD6 (Futurrex, Inc.), and liftoff was performed in n-methyl-2-pyrrolidone at 70° C. Metal deposition was performed using an AJA e-beam evaporator, and atomic layer deposition was performed using a Cambridge Nanotech ALD S100. Continuous monolayer molybdenum disulfide (MoS₂) was synthesized using chemical vapor deposition (CVD). Sulfur (S) powder (MilliporeSigma, 99.98%) and molybdenum trioxide (MoO₃) powder (MilliporeSigma, 99.97%) were used as chemical precursors, and single-crystal sapphire (MTI Corporation, <0001>) was used as the substrate. After growth, monolayer MoS₂ was transferred to the undoped Si substrate through a polycarbonate-assisted transfer process. The MoS₂ monolayer was patterned using a positive resist bilayer of MicroChem PMGI baked at 170° C. and MicroChem S1813 baked at 115° C. MoS₂ was etched by reactive ion etching with a Samco RIE-10NR using 50 sccm Ar at 13.3 Pa and 50 W for 20 sec. Semiconducting CNTs (IsoNanotubes-S 99% purity, Nanolntegris) were vacuum filtered onto a cellulose membrane (VMWP, 0.05 μm pore size, MilliporeSigma) and acetone-bath transferred overnight onto the sample. The CNTs were patterned using S1813 resist, and etched with reactive ion etching using 20 sccm O₂ at 26.5 Pa and 100 W for 10 sec.

Materials characterization and electrical measurements: The thicknesses of the different device layers were characterized by atomic force microscopy (AFM) in ambient conditions using an Asylum Cypher AFM. All electrical measurements were performed in ambient conditions on a Cascade MicroTech semi-automated probe system using a Keithley 4200 semiconductor analyzer.

Support vector machine processing: A support vector machine (SVM) algorithm finds an optimal separating hyperplane by maximizing possible margins between points that belong to different classes. It can be mathematically formulated as a linearly constrained quadratic programming problem as shown in Equation (1):

$${\sum }_{i = 1}^{n} {\sum }_{j = 0}^{n} {λ}_{i} {λ}_{j} K ( {x}_{i} , {x}_{j} ) \geq 0 \qquad ( 2 )$$

subject to the condition in Equation (8) below, where w∈ [symbol] ⁿ gives the normal direction of the hyperplanes and b is a scalar. A dual problem can be equivalently derived with regard to the primal quadratic programming problem, which simplifies the computation. This dual Lagrangian formulation is described in Equation (7) below.

SVM can also be generalized to linearly non-separable applications with the introduction of kernel functions. The kernel functions transform the input data into a higher dimensional Hilbert feature space before performing linear separation. A kernel is essentially a symmetric function K(xᵢ,xⱼ) under a necessary and sufficient condition given by Mercer's theorem:

$${minimize}_{w , b} f ( w , b ) = \frac{1}{2} {‖ w ‖}^{2} \qquad ( 1 )$$

for any datasets x_{1:n}={x₁, . . . , xₙ} and any real numbers λ_{1:n}={λ₁, . . . , λₙ}. In this study, we specifically utilize a Gaussian kernel, also known as radial basis function (RBF) kernel: K(xᵢ,xⱼ)=e^{-∥x}^{ᵢ}^{-x}^{ⱼ}^{∥/2σ}^{²}; a sigmoid kernel: K(xᵢ,xⱼ)=tanh (γ(xᵢ,xⱼ)); and mixed kernels with tunable mixing ratios.

Bayesian optimization: In this study, we use a Bayesian optimization (BO) algorithm for efficient hyperparameter searching. BO is an optimization method that uses prior observations of predefined loss ƒ to determine the next search point. For an input dataset x_{1:n}={x₁, . . . , xₙ}, the loss function ƒ_{1:n}={ƒ(x₁), . . . , ƒ(xₙ)} can be described by a Gaussian process in Equation (3) for computing the prior distribution:

ƒ_{1:n}˜ [symbol] (m(x_{1:n}),K) (3)

where the mean function is m(x_{1:n})=[m(x₁), . . . , m(xₙ)]^{T}, and the n×n covariance kernel matrix K is defined by component in Equation (4):

[K]ᵢⱼ=k(xᵢ,xⱼ) (4)

An acquisition function is a function of the posterior distribution over the loss function ƒ_{1:n}. The next search points are determined by maximizing the acquisition function expected improvement (EI) defined in Equation (5):

EI(x)= [symbol] [max{0,ƒ(x)−ƒ(\hat{x})}] (5)

where \hat{x} is the current optimal hyperparameter set, and the data points to sample in the next iteration are calculated by Equation (6):

x_{new}=arg max EI(x) (6)

Hardware implementations of Gaussian kernels have been attempted using CMOS architectures, including bump circuits and other Gaussian function circuits. However, these approaches require several dozens of devices to generate even simple Gaussian functions, and hyperparameter tuning in mixed Gaussian-sigmoid kernels requires even larger circuits. Previous anti-ambipolar heterojunction transistors do not have sufficient electrostatic control to realize the full hyperparameter tuning of the mixed Gaussian-sigmoid kernels shown in this work. The key innovation at the device level is a precise offset of p-type and n-type components in the heterojunction diode in addition to optimization of the CNT density to electrostatically control the underlying MoS₂ channel through the CNT network without compromising the hole transconductance from the p-type CNTs.

Due to the increasing ubiquity of data-collecting portable electronics and sensors, there is a growing demand for low-power hardware that is capable of performing machine learning algorithms at the point of detection without having to transfer massive amounts of date to the cloud. Analog electronics are promising for such low-power implementations, but fully tunable Gaussian and related nonlinear functions are difficult to generate in simple circuits (e.g., CMOS circuits require close to one hundred devices for a simple mixed Gaussian-sigmoid kernel). In addition, incumbent approaches lack the hyperparameter tunability needed to process large diagnostic datasets such as electrocardiograms (ECGs) from wearable electronics. The present reconfigurable heterojunctions are capable of generating Gaussian, sigmoid, and mixed kernels with full hyperparameter tunability using 100-fold fewer devices than the state-of-the-art.

### Structure of MKH Transistors and Generation of Tunable Mixed Kernels

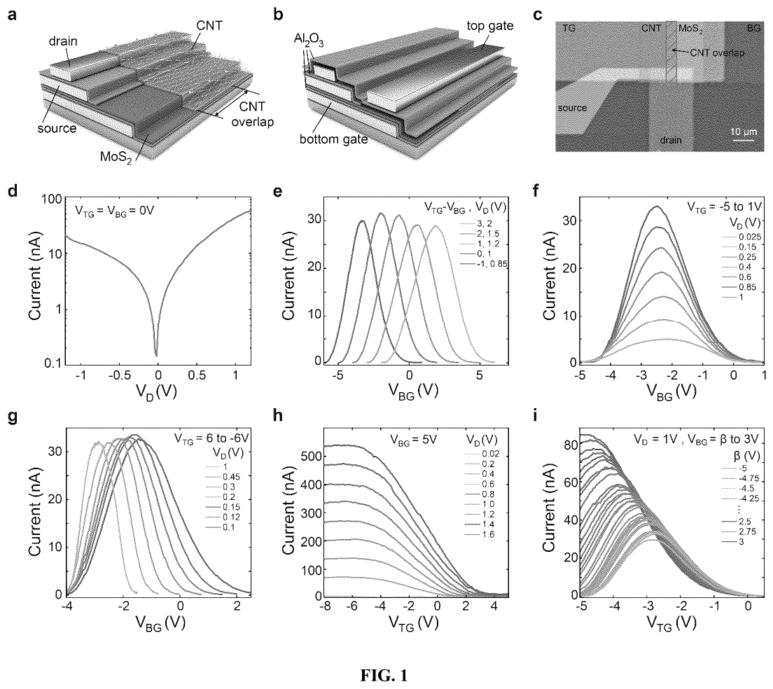

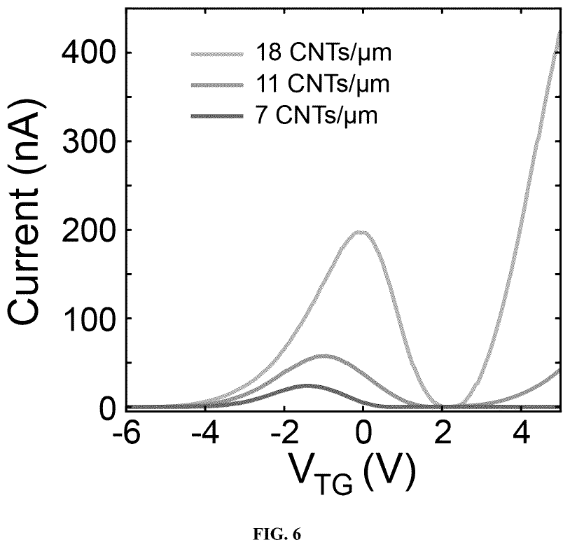

Our MKH transistors are designed to realize rich and distinct functionalities that have not been realized in previous anti-ambipolar devices. First, the semi-vertical geometry of our device design allows for dual-gating of both overlapping and non-overlapping regions of MoS₂ and CNT in the heterojunction (panels a-c of FIG. 1). The overlap region of the MoS₂/CNT heterostructure forms a p-n junction diode (panel d of FIG. 1) with nanomaterial-enabled partial electric-field screening in the overlap region. The overlap region in combination with the MoS₂ and CNT transistors in series in the non-overlapping regions enables highly tunable anti-ambipolar transfer characteristics. Panels e-g of FIG. 1 show charge transport measurements under different dual-gating conditions that yield Gaussian kernel functions with tunable mean panel e of FIG. 1), amplitude (panel f of FIG. 1) and standard deviation (panel g of FIG. 1). Compared with previous literature, the Gaussian behavior in our MKH transistors is both symmetric and shows significant width tunability, which is enabled by the weak screening in the overlap region. Solution-processed CNTs are advantageous for this purpose since their network density can be tuned over a wide range, thus allowing precise control over the degree of screening. In the MKH transistors, a linear density of ˜7 CNTs/μm is used, which avoids the n-type arm in the CNT ambipolar response compared to higher CNT densities (FIG. 6) and provides the optimal level of top-gate screening.

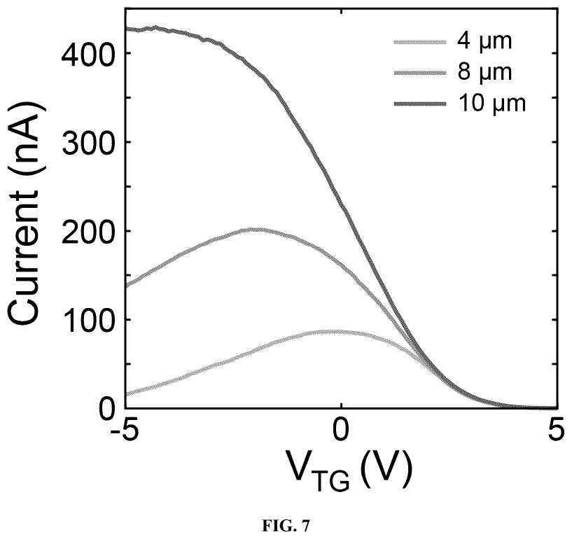

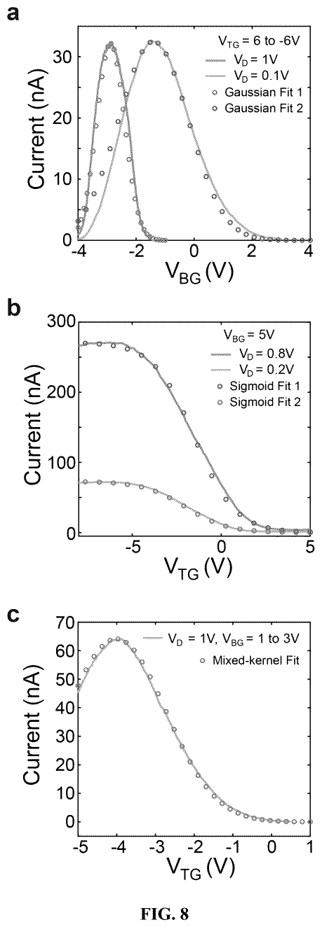

By varying the area of the overlapping region of MoS₂/CNT heterojunction, the MKH transistors can also produce sigmoid kernel functions with tunable slopes by sweeping the top gate (V_{TG}) while keeping the back gate (V_{BG}) fixed at 5 V (panel h of FIG. 1). This additional sigmoid functionality has not been realized in previous anti-ambipolar devices. In the MKH transistors, a 10 μm overlap region of the MoS₂/CNT heterojunction yields optimal sigmoid functions in comparison with smaller overlap sizes (FIG. 7) because it provides improved screening of the top-gate electric field for the MoS₂ film, which minimizes distortion of the sigmoid functions at large negative top-gate biases. Additionally, by tuning V_{BG}, a series of transfer characteristics can be generated that contain both sigmoid and Gaussian characteristics (panel h of FIG. 1), where curve fitting confirms Gaussian and sigmoid mixed-kernel functionality (FIG. 8). This single-device mixed-kernel generation is also unique to our MKH transistors, where the dual-gated architecture coupled with optimized electric-field screening allows for tailored control over the carrier concentrations in the MoS₂, CNT, and overlapping MoS₂/CNT heterojunction regions.

In single-gated anti-ambipolar devices, the lack of hyperparameter tunability of the Gaussian curves limits their utility for kernel generation since hardware implementations would require additional overhead circuity. Lateral anti-ambipolar devices also suffer from higher power consumption, which limits their scalability. Therefore, the reconfigurable MKH transistors reported here have multiple advantages over incumbent anti-ambipolar devices. First, multiple types of kernel functions including Gaussian, sigmoid, and mixed Gaussian and sigmoid curves can be generated in a single MKH device. Second, dynamic reconfigurability of the kernel functions is realized by simply modulating gate fields in the device channel by utilizing the weak screening of atomically thin semiconductors. Third, even without scaling the devices down to the deep submicron limit, the power consumption for MKH transistors to generate Gaussian curves is already comparable to the lowest power consumption that has been experimentally reported in scaled CMOS Gaussian generation circuits. Moreover, the latter CMOS circuit requires more device overhead and complicated designs, which ultimately preclude its use for edge applications. These multifold advantages of MHK transistors for generating mixed kernels enable efficient and effective SVM classification as will be demonstrated below for the specific case of personalized arrhythmia detection from ECG data.

### Mixed-Kernel Support Vector Machine Classification for Arrhythmia Detection

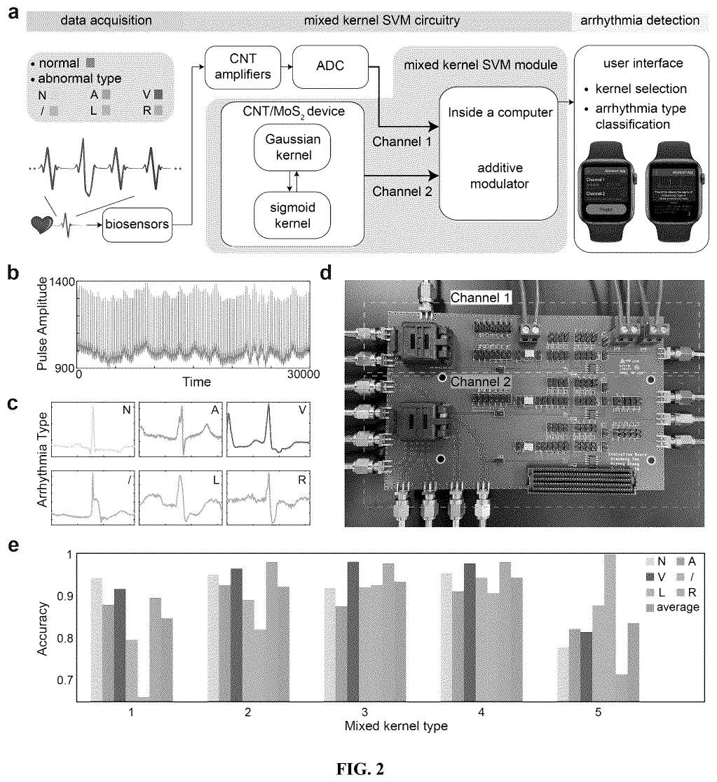

To illustrate the utility and effectiveness of our MKH transistors in practical applications, we explored mixed-kernel SVM classification for personalized arrhythmia detection from ECG data. Our system for real-time arrhythmia detection includes data acquisition, mixed-kernel SVM circuitry for classification, and a user interface (panel a of FIG. 2). Typical ECG input signals are shown in panel b of FIG. 2 for a variety of arrhythmia types. As shown in panel c of FIG. 2, six different types of arrhythmias are considered here: (1) Normal beat (N); (2) Atrial premature beat (A); (3) Premature ventricular contraction (V); (4) Paced beat (/); (5) Left bundle branch block beat (L); (6) Right bundle branch block beat (R). Ambulatory ECG recordings are collected from biosensors, amplified, and preprocessed by analog-to-digital converters (ADCs), which is labeled as the Channel 1 input in panel d of FIG. 2. Two approaches are considered for kernel function generation, which is labeled as the Channel 2 input in panel d of FIG. 2. The first approach uses only one MKH transistor to internally generate tunable Gaussian, sigmoid, and mixed kernels. The second approach uses two MKH transistors that are separately optimized for tunable Gaussian kernels and tunable sigmoid kernels, which are then externally mixed to produce a more complete set of mixed kernels compared to the first approach. The mixing ratio is dynamically tuned by using an additive modulator based on the optimization results. A mixed-kernel SVM module then receives Channel 1 and Channel 2 inputs to perform arrhythmia detection. A final user interface monitors the two channel inputs and displays arrhythmia type classification results.

Different combinations of Gaussian/sigmoid mixed kernels were tested for ECG classification. In particular, five different mixed kernels were generated using one MKH transistor, which correspond to mixed-kernel functions having β values of −5 V, −3 V, −1 V, 1 V, and 3 V in panel i of FIG. 1. Panel e of FIG. 2 shows the classification accuracies for correctly identifying each arrhythmia type (i.e., N, A, V, /, L, and R) out of 10,000 input ECG pulse waveform samples. The mixed kernel with a β value of 1 V yields the highest average arrhythmia detection accuracy with all arrhythmia types being detected with an accuracy at or above the ˜90% level. These experimental results confirm that different mixed-kernel ratios affect the SVM classification accuracy, thus highlighting the importance of dynamic tunability in mixed-kernel hardware implementations.

### Bayesian Optimized Mixed Kernels for Personalized Arrhythmia Detection

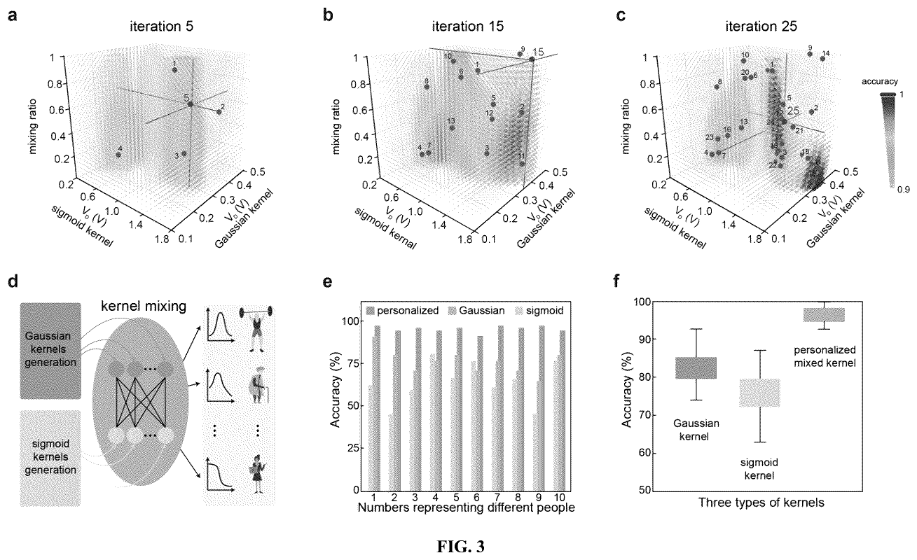

For SVM classification, choosing the optimal kernel is critical for high classification accuracy. Since the optimal selection of hyperparameters can vary greatly for different applications and scenarios, brute force combinatorial optimization is typically impractical, especially for personalized, real-time applications. Therefore, a dynamically reconfigurable mixed-kernel hardware solution needs to be used in conjunction with an efficient hyperparameter optimization strategy. In this regard, Bayesian optimization (BO) is a promising option since it belongs to a class of sequential model-based optimization algorithms that has been shown to outperform random research or grid search in terms of convergence performance. In our case, the hyperparameters for the mixed kernel are optimized iteratively using BO by maximizing the marginal likelihood of arrhythmia detection using a Gaussian process (GP). GP is a generalized Gaussian distribution that is specified by mean and covariance functions, which acts as a prior probability model. The BO process initiates from a random sample in the hyperparameter space. After each BO iteration, the expected improvement (EI)) serves as an acquisition function to determine the next search point in the hyperparameter space. Panels a-c of FIG. 3 show the BO results after 5, 15, and 25 iterations, respectively. After 25 iterations, the search points have converged to the optimal hyperparameter combination, where the highest classification accuracy is achieved (i.e., the red region of panel c of FIG. 3).

Since the BO process enables highly efficient optimization, it is suitable for determining optimal mixed-kernel hyperparameters for personalized or group-based classification. In addition, because BO can accommodate relatively small and noisy datasets, it is appropriate for mobile-friendly arrhythmia detection and related edge-computing use cases. To quantify the effectiveness of this approach, BO-optimized mixed-kernel SVM was used for personalized arrhythmia detection as schematically shown in panel d of FIG. 3. This assessment was carried out by randomizing 100 arrhythmia records from the publicly available MIT-BIH arrhythmia database. These records were used as the input dataset to the mixed-kernel SVM system, after which BO was employed for the mixed-kernel hyperparameter selection for each case. The average classification accuracy over the six arrhythmia types was then calculated, where the results of 10 specific records are provided in panel e of FIG. 3. Compared to the classification results achieved using only Gaussian or only sigmoid kernels, the personalized mixed kernels were better suited for diverse patient datasets, resulting in consistently higher arrhythmia detection accuracy (panel f of FIG. 3).

### Comparing MKH Classification With Conventional CMOS Implementations

In this section, we compare our MKH devices with previous experimentally demonstrated Gaussian/sigmoid mixed-kernel hardware implementations based on conventional CMOS circuits. Early CMOS demonstrations of Gaussian function generation utilized bump circuits, after which CMOS Gaussian function circuits were studied for hardware implementations of machine learning algorithms, smart sensors, and neuromorphic computing systems. While the bump circuit is still the most robust and low-power CMOS method for generating fixed Gaussian curves, scalability issues limit the use of bump circuits for tunable Gaussian function generation. Since a bump circuit generates only one type of Gaussian curve, the generation of tunable Gaussian functions requires cascading multiple bump circuits with each circuit containing CMOS transistors of different channel width and length. Therefore, the number of CMOS transistors quickly increases for fine-grained tunable Gaussian kernels. Alternatively, the additional of extra components to the bump circuit, such as operational transconductance amplifiers and digital-to-analog converters (DACs), can provide tunable Gaussian curves but also introduce additional scalability issues. For example, the operational transconductance amplifier approach requires matching of the channel-length-modulation parameters of short-channel transistors, while the DAC approach has high power consumption (>100 μW) and large footprint (>0.02 mm²) due to the peripheral supporting circuitry. Beyond bump circuits, alternative CMOS circuits that employ absoluters, squarers, and exponentiators also require high power consumption and large footprints. The generation of tunable sigmoid functions with conventional CMOS circuits face similar scalability issues as CMOS tunable Gaussian circuits. While full tunable CMOS mixed kernels can in principle be realized by combining the tunable Gaussian and tunable sigmoid CMOS circuits, the aforementioned impractical scaling issues have precluded any experimental demonstrations to date.

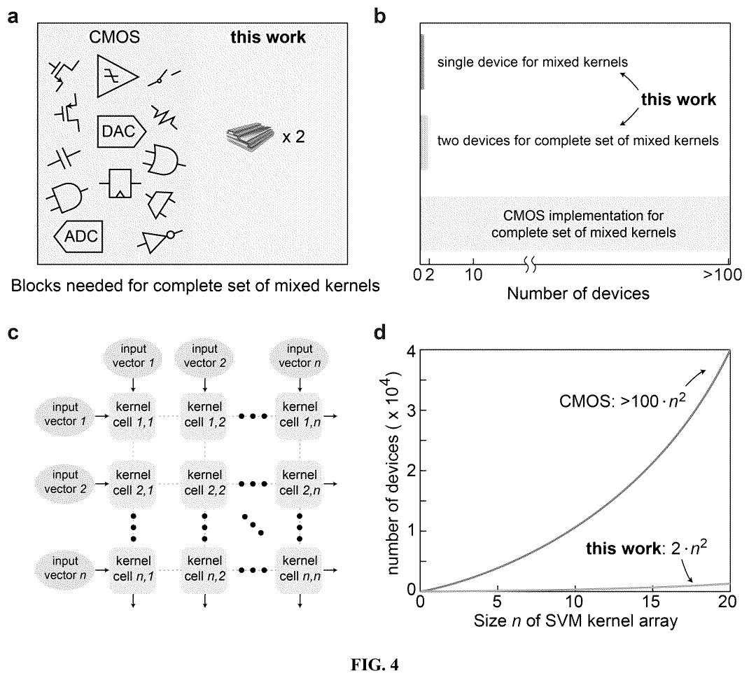

In comparison, MKH transistors feature major advantages over CMOS technology for realizing a complete set of tunable SVM mixed kernels. For example, only two MKH transistors are required for a complete set of tunable mixed kernel generation in contrast to the many building blocks used in CMOS implementations (panel a of FIG. 4). Conservatively, CMOS requires >100 devices to generate a similarly complete set of tunable mixed kernels as can be achieved with only two MKH transistors (panel b of FIG. 4). Consequently, the MKH approach provides a clear footprint advantage compared to CMOS for tunable mixed kernel generation.

The scaling advantage of MKH transistors becomes even more evident when implementing an n×n kernel matrix. Mathematically, finding the primal SVM optimization is equivalent to solving a Lagrangian dual problem as defined in Equation (7) by training n-input vectors xᵢ:

$${max}_{α} {\sum }_{i = 1}^{n} {α}_{i} - \frac{1}{2} {\sum }_{i , j = 1}^{n} {α}_{i} {α}_{j} {y}_{i} {y}_{j} κ ( {x}_{i} , {x}_{j} ) \\ s . t . {α}_{i} \geq 0 , \& {\sum }_{i = 1}^{n} {α}_{i} {y}_{j} = 0 , i = 1 , 2 , … , n \qquad ( 7 )$$

where αᵢ is the Lagrange multiplier corresponding to the inequality constraints in Equation (8):

yᵢ(w^{T}xᵢ+b)≥1,i=1,2, . . . , n (8)

Here, yᵢ is the true classification label corresponding to xᵢ, and κ(xᵢ,xⱼ) is the kernel function operation on xᵢ and xⱼ after substituting the Karush-Kuhn-Tucker (KKT) conditions into the primal Lagrangian. Correspondingly, SVM hardware needs to implement an n×n kernel matrix to calculate the kernel function operation κ(xᵢ,xⱼ) for each pair of input vectors (panel c of FIG. 4). Each kernel cell in this matrix needs to generate a complete set of mixed kernels by having at least 100 CMOS devices or equivalently only two MKH transistors, leading to the plot in panel d of FIG. 4 that compares the total number of devices required for SVM hardware with personalized kernel functionality. In applications where the hardware footprint needs to be minimized (e.g., wearable electronics), the MKH approach has clear advantages over conventional CMOS.

Finally, the MKH approach has reduced power consumption compared to CMOS for mixed-kernel SVM hardware. The tunable Gaussian, sigmoid, and mixed kernels generated by MKH transistors (panels e-i of FIG. 1) only require tens of nanowatts of power (e.g., the estimated power consumption in panel e of FIG. 1 is 30 nA×2 V=60 nW). In contrast, tunable mixed kernels generated using conventional CMOS circuits require tens of microwatts to milliwatts depending on the specific architecture. This power consumption difference is particularly important when implementing an n×n kernel matrix.

### Conclusions

By tailoring the degree of electric-field screening through control over CNT density and overlap area, dual-gated MoS₂/CNT heterojunctions enable the design of MKH transistors with tunable Gaussian, sigmoid, and mixed kernel functionality. The self-aligned, semi-vertical device geometry implies that a complete set of mixed Gaussian/sigmoid kernels can be achieved simply by varying the biases to the top and bottom gates. Using MKH transistors, a mixed-kernel SVM platform for arrhythmia detection was demonstrated, where optimal Gaussian/sigmoid hyperparameters and mixed-kernel ratios were determined by BO to achieve exceptionally high arrhythmia detection accuracies. Moreover, MKH transistors are amenable to personalized kernels that enable arrhythmia detection accuracies approaching 95% for diverse patient profiles. This MKH approach offers clear advantages over conventional CMOS implementations including simpler circuit designs, smaller footprints, and lower power consumption. In this manner, MKH transistors have the potential to be widely applicable to SVM classification in diverse wearable and edge applications.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described to explain the principles of the invention and their practical application to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Noble, W. S. What is a support vector machine? Nature Biotechnology 24, 1565-1567, (2006).
[2]. Cervantes, J., Garcia-Lamont, F., Rodríguez-Mazahua, L. & Lopez, A. A comprehensive survey on support vector machine classification: Applications, challenges and trends. Neurocomputing 408, 189-215, (2020).
[3]. Genov, R. & Cauwenberghs, G. Kerneltron: support vector “machine” in silicon. IEEE Transactions on Neural Networks 14, 1426-1434, (2003).
[4]. Devikanniga, D., Ramu, A. & Haldorai, A. Efficient diagnosis of liver disease using support vector machine optimized with crows search algorithm. EAI Endorsed Transactions on Energy Web 7, e10, (2020).
[5]. Wang, H., Zheng, B., Yoon, S. W. & Ko, H. S. A support vector machine-based ensemble algorithm for breast cancer diagnosis. European Journal of Operational Research 267, 687-699, (2018).
[6]. Ahmad, I., Basheri, M., Iqbal, M. J. & Rahim, A. Performance comparison of support vector machine, random forest, and extreme learning machine for intrusion detection. IEEE Access 6, 33789-33795, (2018).
[7]. Afifi, S. M., GholamHosseini, H. & Sinha, R. Hardware implementations of SVM on FPGA: A state-of-the-art review of current practice. International Journal of Innovative Science, Engineering & Technology 2, 733-752, (2015).
[8]. Shoeb, A. H. & Guttag, J. V. Application of machine learning to epileptic seizure detection. In ICML 975-982 (PMLR, 2010).
[9]. Bin Altaf, M. A. & Yoo, J. A 1.83 J/classification, 8-channel, patient-specific epileptic seizure classification SoC using a non-linear support vector machine. IEEE Transactions on Biomedical Circuits and Systems 10, 49-60, (2016).
[10]. Kang, K. & Shibata, T. An on-chip-trainable gaussian-kernel analog support vector machine. IEEE Transactions on Circuits and Systems I: Regular Papers 57, 1513-1524, (2010).
[11]. Zhang, R. & Shibata, T. Fully parallel self-learning analog support vector machine employing compact gaussian generation circuits. Japanese Journal of Applied Physics 51, 04DE10, (2012).
[12]. Alimisis, V., Gourdouparis, M., Dimas, C. & Sotiriadis, P. P. A 0.6 V, 3.3 nW, adjustable gaussian circuit for tunable kernel functions. In 2021 34th SBC/SBMicro/IEEE/ACM Symposium on Integrated Circuits and Systems Design (SBCCI) 1-6 (IEEE, 2021).
[13]. Vrtaric, D., Ceperic, V. & Baric, A. Area-efficient differential gaussian circuit for dedicated hardware implementations of gaussian function based machine learning algorithms. Neurocomputing 118, 329-333, (2013).
[14]. Reda Mohamed, A., Qi, L., Li, Y. & Wang, G. A generic nano-watt power fully tunable 1-D gaussian kernel circuit for artificial neural network. IEEE Transactions on Circuits and Systems II: Express Briefs 67, 1529-1533, (2020).
[15]. Sangwan, V. K. & Hersam, M. C. Neuromorphic nanoelectronic materials. Nat Nanotechnol 15, 517-528, (2020).
[16]. Sangwan, V. K. et al. Self-aligned van der Waals heterojunction diodes and transistors. Nano Letters 18, 1421-1427, (2018).
[17]. Sebastian, A., Pannone, A., Subbulakshmi Radhakrishnan, S. & Das, S. Gaussian synapses for probabilistic neural networks. Nature Communications 10, 4199, (2019).
[18]. Beck, M. E. et al. Spiking neurons from tunable gaussian heterojunction transistors. Nature Communications 11, 1565, (2020).
[19]. Duong, D. L., Lee, S. M. & Lee, Y. H. Origin of unipolarity in carbon nanotube field effect transistors. Journal of Materials Chemistry 22, 1994-1997, (2012).
[20]. Wang, K.-C. et al. Atomic-level charge transport mechanism in gate-tunable anti-ambipolar van der Waals heterojunctions. Applied Physics Letters 118, 083103, (2021).
[21]. Kim, C. H., Hayakawa, R. & Wakayama, Y. Fundamentals of organic anti-ambipolar ternary inverters. Advanced Electronic Materials 6, 1901200, (2020).
[22]. Wu, E. et al. Photoinduced doping to enable tunable and high-performance anti-ambipolar MoTe₂/MoS₂ heterotransistors. ACS Nano 13, 5430-5438, (2019).
[23]. Kobashi, K., Hayakawa, R., Chikyow, T. & Wakayama, Y. Multi-valued logic circuits based on organic anti-ambipolar transistors. Nano Letters 18, 4355-4359, (2018).
[24]. Li, Y. et al. Anti-ambipolar field-effect transistors based on few-layer 2D transition metal dichalcogenides. ACS Applied Materials & Interfaces 8, 15574-15581, (2016).
[25]. Song, M. H., Lee, J., Cho, S. P., Lee, K. J. & Yoo, S. K. Support vector machine based arrhythmia classification using reduced features. International Journal of Control, Automation and Systems 3, 571-579, (2005).
[26]. Asl, B. M., Setarehdan, S. K. & Mohebbi, M. Support vector machine-based arrhythmia classification using reduced features of heart rate variability signal. Artificial Intelligence in Medicine 44, 51-64, (2008).
[27]. Shahriari, B., Swersky, K., Wang, Z., Adams, R. P. & De Freitas, N. Taking the human out of the loop: A review of Bayesian optimization. Proceedings of the IEEE 104, 148-175, (2015).
[28]. Moody, G. B. & Mark, R. G. The impact of the MIT-BIH arrhythmia database. IEEE Engineering in Medicine and Biology Magazine 20, 45-50, (2001).ja
[29]. Goldberger, A. L. et al. PhysioBank, PhysioToolkit, and PhysioNet: Components of a new research resource for complex physiologic signals. Circulation 101, e215-e220, (2000).
[30]. Delbruck, T. ‘Bump’ circuits for computing similarity and dissimilarity of analog voltages. In IJCNN-91-Seattle International Joint Conference on Neural Networks 475-479 (IEEE, 1993).
[31]. Verleysen, M., Thissen, P., Voz, J.-L. & Madrenas, J. An analog processor architecture for a neural network classifier. IEEE Micro 14, 16-28, (1994).
[32]. Nam, M. & Cho, K. Implementation of real-time image edge detector based on a bump circuit and active pixels in a CMOS image sensor. Integration 60, 56-62, (2018).
[33]. Payvand, M. & Indiveri, G. Spike-based plasticity circuits for always-on on-line learning in neuromorphic systems. In 2019 IEEE International Symposium on Circuits and Systems (ISCAS) 1-5 (IEEE, 2019).
[34]. Lu, J., Yang, T., Jahan, M. & Holleman, J. Nano-power tunable bump circuit using wide-input-range pseudo-differential transconductor. Electronics Letters 50, 921-923, (2014).
[35]. Alimisis, V., Gourdouparis, M., Gennis, G., Dimas, C. & Sotiriadis, P. P. Analog gaussian function circuit: Architectures, operating principles and applications. Electronics 10, 2530, (2021).
[36]. Youssefi, B., Leigh, A. J., Mirhassani, M. & Wu, J. Tunable neuron with PWL approximation based on the minimum operator. IEEE Transactions on Circuits and Systems II: Express Briefs 66, 387-391, (2018).
[37]. Yan, X., Qian, J. H., Sangwan, V. K. & Hersam, M. C. Progress and challenges for memtransistors in neuromorphic circuits and systems. Advanced Materials, 34, 2108025, (2022).
[38]. Yiguang He et al., A kind of analog-circuit fault diagnosis method based on broad sense multi-kernel support vector machine. China Patent No. CN105548862B, Feb. 5, 2019.
[39]. Mark C. Hersam et al., Gate-tunable p-n heterojunction diode, and fabrication method and application of same. U.S. Pat. No. 9,972,799B2, May 15, 2018.
[40]. Deep M. Jariwala et al., System and method for anti-ambipolar heterojunctions from solution-processed semiconductors. U.S. Pat. No. 10,491,206B2, Nov. 26, 2019.
[41]. Joel W. Burdick et al., Neurostimulator devices using a machine learning method implementing a gaussian process optimization. U.S. Pat. No. 9,931,508B2, Apr. 3, 2018.
[42]. Jariwala, D. et al. Hybrid, Gate-Tunable, van der Waals p-n Heterojunctions from Pentacene and MoS₂. Nano Letters 16, 497-603, (2016).
[43]. Jariwala, D. et al. Large-Area, Low-Voltage, Antiambipolar Heterojunctions from Solution-Processed Semiconductors. Nano Letters 15, 416-421, (2015).
[44]. Jariwala, D. et al. Gate-tunable carbon nanotube-MoS2 heterojunction p-n diode. Proceedings of the National Academy of Sciences 110, 18076, (2013).

What is claimed is:

1. A mixed-kernel heterojunction (MKH) transistor, comprising:
a monolayer film formed of an atomically thin material, and a network of carbon nanotubes (CNTs) vertically stacked over the monolayer film to define an overlap region of the CNT network with the monolayer film, and non-overlap regions of the monolayer film and the CNT network, wherein the overlap region is a mixed-kernel van der Waals heterojunction.

2. The MKH transistor of claim 2, further comprising:
a bottom gate electrode, a top gate electrode, a source electrode, a drain electrode, a first dielectric layer, a second dielectric layer, and a third dielectric layer, wherein
the bottom gate electrode is formed on a substrate;
the first dielectric layer is formed on the bottom gate electrode;
the monolayer film is formed on the first dielectric layer;
the source electrode is formed on a part of the monolayer film;
the second dielectric layer is formed on the source electrode;
the drain electrode is formed on the second dielectric layer on the top of the source electrode;
the CNT network is formed on the drain electrode and the monolayer film to define the overlap region comprising the CNT network and the monolayer film, and the non-overlap regions each of which comprising a respective one of the CNT network and the monolayer film;
the third dielectric layer is formed on the CNT network, the monolayer film and the drain electrode over the substrate; and
the top gate electrode is formed on the third dielectric layer and overlapping with the overlap region and the non-overlap regions.

3. The MKH transistor of claim 2, wherein the atomically thin material comprises a two-dimensional (2D) semiconductor material.

4. The MKH transistor of claim 3, wherein the 2D semiconductor material comprises MoS₂, MoSe₂, WS₂, WSe₂, InSe, GaTe, black phosphorus (BP), or related 2D materials.

5. The MKH transistor of claim 2, wherein the bottom and top gate electrodes and the source and drain electrodes comprise a same conductive material or different conductive materials.

6. The MKH transistor of claim 5, wherein each of the bottom and top gate electrodes and the source and drain electrodes is formed of gold (Au), titanium (Ti), aluminum (Al), nickel (Ni), chromium (Cr), or other conductive materials.

7. The MKH transistor of claim 2, wherein the first, second and third dielectric layers comprise a same dielectric material or different dielectric materials.

8. The MKH transistor of claim 7, wherein each of the first, second and third dielectric layers is formed of Al₂O₃, HfO₂, ZrO₂, ZnO, SiO₂, or dielectrics including alumina, hafnia, or zirconia.

9. The MKH transistor of claim 2, wherein the monolayer film comprises a monolayer MoS2 grown by chemical vapor deposition (CVD), mechanical exfoliation, metal-organic chemical vapor deposition (MOCVD), or atomic layer deposition (ALD) as an n-type material, and the CNT network comprises solution-processed semiconducting CNT thin film as a p-type material.

10. The MKH transistor of claim 9, wherein the overlap region in combination with the MoS₂ and CNT transistors in series in the non-overlapping regions enables highly tunable anti-ambipolar transfer characteristics.

11. The MKH transistor of claim 9, wherein the overlap region of the MoS₂/CNT heterostructure forms a p-n junction diode with nanomaterial-enabled partial electric-field screening in the overlap region.

12. The MKH transistor of claim 11, wherein the overlap region of the MoS₂/CNT heterostructure controls the degree of electric-field screening of the top and bottom gates.

13. The MKH transistor of claim 12, wherein Gaussian kernel functions with tunable mean, amplitude and standard deviation are yielded under different dual-gating conditions.

14. The MKH transistor of claim 13, wherein the Gaussian behavior is both symmetric and shows significant width tunability, which is enabled by the weak screening in the overlap region.

15. The MKH transistor of claim 9, wherein the network density of the solution-processed CNTs is tunable over a wide range, thereby allowing precise control over the degree of screening.

16. The MKH transistor of claim 15, wherein the network density comprises a linear density of about 7 CNTs/μm, which avoids the n-type arm in the CNT ambipolar response compared to higher CNT densities and provides the optimal level of top-gate screening.

17. The MKH transistor of claim 9, wherein by tailoring the degree of electric-field screening through control over CNT density and overlap area, dual-gated MoS₂/CNT heterojunctions enable the MKH transistor with tunable Gaussian, sigmoid, and mixed kernel functionality.

18. The MKH transistor of claim 17, wherein a 10 μm overlap region of the MoS₂/CNT heterojunction yields optimal sigmoid functions in comparison with smaller overlap sizes.

19. The MKH transistor of claim 2, wherein precise control over electric-field screening in MKH transistor enables the generation of a complete set of fine-grained Gaussian, sigmoid, and mixed-kernel functions using only a single device.

20. The MKH transistor of claim 2, wherein the MHK transistor for generating mixed kernels enables efficient and effective SVM classification for personalized arrhythmia detection from electrocardiogram (ECG) data.

21. The MKH transistor of claim 2, being amenable to personalized kernels that enable arrhythmia detection accuracies approaching 95% for diverse patient profiles.

22. The MKH transistor of claim 2, wherein in conjunction with Bayesian optimization, the MKH transistor provides effective and efficient hyperparameter searching, which further enhances classification performance.

23. The MKH transistor of claim 2, being configured such that the number of circuit elements for mixed-kernel SVM is reducible by approximately two orders of magnitude, thereby enabling high classification accuracy in a scalable and energy-efficient manner.

24. The MKH transistor of claim 2, wherein the self-aligned, semi-vertical device geometry enables to achieve a complete set of mixed Gaussian/sigmoid kernels simply by varying the biases to the top and bottom gates.

25. A circuit, comprising at least one MKH transistor according to claim 1.

26. A system for real-time arrhythmia detection, comprising:
a data acquisition for ambulatory ECG recordings;
a mixed-kernel support vector machine (SVM) circuitry for classification, wherein the mixed-kernel SVM circuitry comprises a mixed-kernel heterojunction (MKH) transistor device, and a mixed-kernel SVM module coupled with the data acquisition and the MKH transistor device for receiving inputs therefrom to perform arrhythmia detection; and
a user interface coupled with the mixed-kernel SVM circuitry for monitoring the inputs and displaying arrhythmia types of the classification.

27. The system of claim 26, wherein the ambulatory ECG recordings are collected from biosensors, amplified, and preprocessed by analog-to-digital converters (ADCs).

28. The system of claim 26, wherein the MKH transistor device comprises a single MKH transistor to internally generate tunable Gaussian, sigmoid, and mixed kernels.

29. The system of claim 26, wherein the MKH transistor device comprises two MKH transistors that are separately optimized for tunable Gaussian kernels and tunable sigmoid kernels, which are then externally mixed to produce a complete set of mixed kernels.

30. The system of claim 29, wherein the mixing ratio is dynamically tuned by using an additive modulator based on the optimization results.

31. The system of claim 26, wherein hyperparameters for the mixed kernel are optimized iteratively using Bayesian optimization (BO) by maximizing the marginal likelihood of arrhythmia detection using a Gaussian process (GP), wherein the GP is a generalized Gaussian distribution that is specified by mean and covariance functions, which acts as a prior probability model.

32. The system of claim 31, wherein the BO process initiates from a random sample in the hyperparameter space, wherein after each BO iteration, an expected improvement (EI) serves as an acquisition function to determine the next search point in the hyperparameter space, and repeats until the search points have converged to an optimal hyperparameter combination in which the highest classification accuracy is achieved.

33. The system of claim 32, wherein BO-optimized mixed-kernel SVM is used for personalized arrhythmia detection.

34. The system of claim 26, wherein in an SVM hardware implementation, the scalability of the MKH transistors comprises an n×n kernel matrix.

35. The system of claim 34, wherein each kernel cell in the n×n kernel matrix needs to generate a complete set of mixed kernels by only two MKH transistors.

36. The system of claim 26, wherein the MKH approach has reduced power consumption compared to CMOS for mixed-kernel SVM hardware. The tunable Gaussian, sigmoid, and mixed kernels generated by MKH transistors only require tens of nanowatts of power.
